(19) United States
(12) Patent Application Publication
(10) Pub. No.: US 2005/0048132 A1
(43) Pub. Date: Mar. 3, 2005

(54) Use of hydrochloric acid in the preparation of a medicament for the treatment of tumour

(76) Inventor: Weijian Feng, Beijing (CN)

Correspondence Address: BARNES & THORNBURG, P.O. BOX 2786, CHICAGO, IL, 60690-2786, US

(21) Appl. No.: 10/500,080

(22) PCT Filed: May 17, 2002
(86) PCT No.: PCT/CN02/00335

(30) Foreign Application Priority Data
Dec. 24, 2001 (CN) 01144650.1

(51) Int. Cl. A61K 33/18

(52) U.S. Cl. 424/666

(57) ABSTRACT

The use of hydrochloric acid in the pharmaceutical field for tumorous tissue coagulation and necrosis is disclosed. The effect hydrochloric acid is preferable to the effects of other medicaments. Hydrochloric acid can be used for treating any noumenal tumour, including primary therioma, metastatic theioma, benign tumour, and also for destructing nerve to treat cancerous pain. Hydrochloric acid can be used for the manufacture of a medicament for the curative treatment of tumors.

### BACKGROUND

This invention relates to the use of hydrochloric acid and the use of hydrochloric acid in the preparation of medicaments for the treatment of tumors and to relieve cancerous pain.

The treatment of tumors, including malignant tumors, is carried out by the removal of tumors through surgical operation. In the recent 20 years, some non-surgical local therapeutic treatments have been developed—the aims of these treatments are to cause necrosis of the treated tumor, while the normal tissues are minimally injured. Among the non-operative physical treatment means, X-ray knife uses radiation to treat tumors with localized irradiation. Ultrasonic focusing knife makes use of ultrasonic waves to heat and damage the tumor. Microwave radio-frequency uses heat to coagulate tumors and argon-helium knife uses the gasses of argon and helium to freeze and heat tumors so that the tumors can be coagulated. All these methods, by means of guided localization, can coagulate and kill the tumor tissues. Some of the disadvantages of these methods include: use of expensive equipment, high costs, and some of the methods may bring about the problem of remaining “blind region” because they use the technique of “multi-needles” or “the multi-points combination effects”. Some of the methods can not make a spherical coagulating necrotic region that is not in accordance with the phenomenon that generally, a tumor grows in a spherical shape, so the range of treatment is limited. When coagulation cannot be completed, the tumor recurs easily.

Chemical treatments of tumors are to administer intratumoral injections of chemical drugs that act as agents of ablation to promote the coagulation and necrosis of tumors, so that tumors are melted and absorbed by non-surgical treatments. Examples of these methods are injections of chemotherapeutic drugs, absolute alcohol, or acetic acid.

The effects of injecting chemotherapeutic drugs locally are poor and therefore, this technique is not used commonly in clinical practice. Injecting absolute alcohol to coagulate a tumor to treat liver cancer is practiced clinically. The effects of using absolute alcohol to treat liver cancer are better than that of using chemotherapeutic drugs, but when absolute alcohol is used, the coagulating range is small and the therapeutic effects are not stable. Further, alcohol intoxication occurs easily if high dosages of alcohol are used. Ohnishi et al., disclosed the method of using 50% acetic acid to melt and dissipate the tumors firstly in the article “Small hepatocellular carcinoma: treatment with US-guided intertumoral injection of acetic acid” published in Radiology (193, 747-752; 1994). The therapeutic effect of acetic acid is three times as high as that of alcohol, but the border of treated area is not clear. The coagulating necrotic region can not form a globular shape, so the optimum effect of the treatment is difficult to achieve and the surrounding tissues are easily injured. During the treatment, a heavy irritating smell is produced and therefore, the method can not be used widely in clinical practice. There is need for the development of new chemical agents of cytoclasis and melting for tumor, which produce therapeutic effects, accurate, easily controllable, and spherical coagulating range. The new agent should be convenient to use, low cost, and not cause adverse reactions in human bodies.

The inventor of this disclosure, when culturing tumor cell in vitro, discovered that gastric juice had the effect of damaging cancer cells. He further discovered that the effect was produced mainly by the action of hydrochloric acid.

### SUMMARY OF THE DISCLOSURE

An object of this disclosure is to provide a new use of hydrochloric acid is the use of hydrochloric acid in the manufacture of a medicament for the treatment of tumors.

This disclosure relates to the use of hydrochloric acid for the manufacture of a medicament for the treatment of tumors—the tumors include malignant and benign tumors.

This disclosure further relates to the use of hydrochloric acid for the manufacture of a medicament for the treatment of tumors. The malignant tumors are cancers of the liver, lungs, kidney, breast, or their metastatic cancers, such as metastatic carcinoma of the adrenal gland, or the brain.

Another object of this disclosure is to provide a new use of hydrochloric acid for the manufacture of analgesics for relieving cancerous pain.

The concentration of hydrochloric acid used to manufacture the medicament for the treatment of tumors is about 1.8-36 wt %, the amount used is about 0.05-5 ml; a preferred concentration is about 3.6-25.2 wt %, a preferred amount used is about 0.1-4.5 ml; and another preferred concentration is about 18 wt %, another preferred amount used is 0.5-3.0 ml.

The hydrochloric acid of this disclosure used to manufacture tumor-treating medicament is injected slowly into the tumor tissue. By the action of dehydration and protein coagulation of hydrochloric acid, the tumor tissue is induced to coagulate and become necrotic. After a period of time, the coagulating necrotic tissues are absorbed by the organism.

To treat liver cancer, the puncture needle, guided by B-type ultrasonography or CT, is guided to puncture the center of liver tissue. The hydrochloric acid, delivered via an automatic microinjecting pump, is then slowly injected into the tumor tissue. At the same time, 5% sodium bicarbonate solution is administered quickly through intravenous infusion to neutralize the blood. The hydrochloric acid produces coagulation and necrosis of the tumor. The necrotic tissues are then absorbed by the organism after a period of time.

The hydrochloric acid used in the manufacture of a medicament for the treatment of tumors of the present invention is an analytically pure hydrochloric acid available in the market. It has a molecular weight of 36.46, and the hydrogen chloride content is 36-38 wt %. It is a colorless solution with slightly offensive smell, being soluble in water and able to mix with water in any proportion to make diluted hydrochloric acid. The processes for preparing the hydrochloric acid of the present invention is: in asepsis, the analytically pure hydrochloric acid available from the market is diluted with sterile water for injection into different concentrations needed, such as 1.8%, 3.6%, 7.2%, 10.8%, 14.4%, 18%, and 25.2%; and the solutions of different concentrations are stored in different bottles for use. Alternatively, the analytically pure hydrochloric acid (pure hydrochloric acid, 36.4%) is stored in bottles to be used. The amount of hydrochloric acid injected is decided according to the size of the tumor.

In the present disclosure, the hydrochloric acid had the effects of coagulating tissues and tumors by the pathological experiments of pig liver in vivo and in vitro, pig lungs in vivo, tumors of rats in vivo, human liver cancer, and human pulmonary cancer. By comparing the effects of hydrochloric acid, acetic acid and absolute alcohol separately in coagulating pig liver and sarcoma of rats, the effect of coagulating tissues and tumors produced by hydrochloric acid was better than that of acetic acid or absolute alcohol.

Slow injection of hydrochloric acid into a tumor is safe by the pathological experiments in coagulating the skin of guinea pigs with hydrochloric acid and by the observation of toxic actions of hydrochloric acid on pigs in this invention. In order to neutralize a very small amount of hydrochloric acid that might enter into blood circulation, 5% sodium bicarbonate solution is administered quickly through intravenously infusion to neutralize the blood when the diluted hydrochloric acid was injected slowly. Sodium bicarbonate has an antagonistic action on the coagulating effects of hydrochloric acid, and sodium bicarbonate can neutralize the excess amount of hydrochloric acid.

As disclosed herein, hydrochloric acid with different concentrations used in different doses can coagulate tumors of different sizes. Generally, when hydrochloric acid solutions of different concentrations (e.g. 1.8%-36 wt %) are injected in the same dose (X ml), or hydrochloric acid solution of the same concentration (Y %) is injected in different doses (e.g. 0.05 ml -3 ml), tumors less than 5 cm in size can be coagulated; (X refers to any value between 0.05-3 ml, Y refers to any value between 1.8 wt % -36 wt %).

In an embodiment, a tumor with a diameter of less than 3 cm can be cured by injecting 0.5-3 ml of 18 wt % hydrochloric acid solution once.

When cultivation of cancer cells in vitro, together with gastric juice, was performed, it was found that gastric juice destroyed cancer cells. Further experimentation proved that the destruction was due to gastric acid (diluted hydrochloric acid). Hydrochloric acid produces definite effects of tissue dehydration and protein denaturation that can lead to the death of cancer cells. Hence it is possible to use HCl as a chemical ablation agent to coagulate tumor. Also, because hydrochloric acid is a main component of human gastric juice, when hydrochloric acid was injected slowly into the tumor tissue, it diffused slowly into the tissue, coagulated the tissue, and damaged the tumor. General toxic reaction will not occur during the coagulation of human or animal tissue. Moreover, in normal tissues, there are many large blood vessels allowing the injected solution can be easily shunted. However, in tumor tissues, there are more capillary blood vessels, and when injected slowly at an even rate, the hydrochloric acid solution can be evenly distributed in the pathological region. Therefore, the hydrochloric acid injected can coagulate tumor tissue. On the basis of many experiments carried out in vivo and in vitro, the medicament made of hydrochloric acid for the treatment of tumors can inactivate the cancer cells in situ and cure tumors.

The mechanism of hydrochloric acid inactivating cancer tissue is the same as that of strong acid acting on chemical bums. The mechanism is mainly protein denaturation and tissue dehydration.

The effects of coagulating necrosis of tissues produced by injecting hydrochloric acid were definite, safe and reliable. At the same time, it was observed that when hydrochloric acid of a concentration less than 18% was injected locally, the capsule of liver was not damaged, showing that the fibrous tissue had a relatively high tolerance to hydrochloric acid. Generally tumors have fibrous membranes, so the osmosis of hydrochloric acid into the surrounding normal tissues is inhibited and injury to normal tissues potentially caused by hydrochloric acid is reduced. Further, during the use of HCl, in order to prevent excess hydrochloric acid from spreading to normal tissues and causing injuries, 5% sodium bicarbonate solution was administered at the same time through intravenous infusion to neutralize the blood. An alkaline drug, such as sodium bicarbonate, was injected locally to the pathologically changed region for detoxification.

The invention may best be understood by reference to the following description taken in conjunction with the accompany drawing and the embodiments to show the use of hydrochloric acid for the manufacture of a medicament for the treatment of tumors and relief cancerous pain.

### BRIEF DESCRIPTION OF THE DRAWINGS

FIG. 1A is a schematic diagram depicting the coagulation of pig liver in vitro resulting from the action of 0.5 ml of 18 wt % hydrochloric acid.

FIGS. 1B, 1C and 1D are schematic diagrams illustrating respectively the effects of coagulating pig liver in vitro by 0.5 ml of 50% acetic acid, 0.5 ml of 100% absolute alcohol, and micro-wave 60 w for 60 s.

FIGS. 2A-2C are schematic diagrams illustrating the results obtained after the pathologic experiment of 1 week, 4 weeks, and 12 weeks respectively by using 1 ml of 3.6 wt % hydrochloric acid to coagulate pig liver in vivo.

FIGS. 3A and 3B are schematic diagrams illustrating respectively the microscopic histopathological changes of pig liver after 1 week and 4 weeks by taking 1 ml of 3.6 wt % of hydrochloric acid to coagulate the pig liver in vivo.

FIGS. 4A and 4B are schematic diagrams illustrating respectively the anatomical changes of pig lungs observed at 1 week and 4 week after using 1 ml of 3.6 wt % hydrochloric acid to coagulate the pig lungs in vivo.

FIGS. 5A and 5B are schematic diagrams illustrating respectively the microscopic histopathological changes in pig lungs found at 1 week and 4 weeks after using 1 ml of 3.6 wt % hydrochloric acid to coagulate the pig lungs in vivo.

FIGS. 6A and 6B are schematic diagrams illustrating respectively the anatomical changes and microscopic histopathological changes of sarcoma of rat found at 24 hours after injecting 1 ml of 18 wt % hydrochloric acid to the rat with sarcoma.

FIG. 7 is schematic diagram illustrating the result of a comparison between the coagulating effects produced by 18 wt % hydrochloric acid, 50% acetic acid, and absolute alcohol (0.05 ml each) on sarcoma of rat.

FIGS. 8A-8D are schematic diagrams illustrating respectively the CT change and B-type ultrasonographic change found immediately after, and 24 hours after, CT-guided subcutaneous injection of 2 ml of 18% hydrochloric acid to coagulate liver cancer.

FIG. 9 is schematic diagram illustrating the results of the sensitive index—α-fetoglobulin—determined before and after an injection of 1 ml of 18 wt % hydrochloric acid was given to a patient with liver cancer.

FIGS. 10A-10C are schematic diagrams illustrating respectively the CT changes immediately after and 3 days after the injection of 1 ml of 18 wt % hydrochloric acid to coagulate lung cancer, and the pathological and cytological changes of needle-puncture after the injection.

### DETAILED DESCRIPTION OF THE DISCLOSURE

A. The Pathological Experiments on the Coagulating Effect of Hydrochloric Acid on Pig Liver in vitro.

Thirty pig livers purchased from the market were divided into 10 groups, 3 in each group. A 22G puncture needle was punctured to the center of the liver tissue of each pig's liver, and connected with an automatic micro-injecting pump. One milliliter (1 ml) of hydrochloric acid solutions, (the concentrations being 1.8 wt %, 3.6 wt %, 7.2 wt %, 10.8 wt %, 14.4 wt %, 18 wt %, 25.2 wt %), and 1 ml , 0.5 ml, 0.1 ml respectively, of analytical pure hydrochloric acid were injected slowly to the pigs of different groups, at a speed of 0.3 ml/min. The gross specimens were observed and the average value of the diameters of coagulated areas was determined. The findings obtained immediately after injection were as follows: among the dark red liver tissue, with the puncturing point as the center, a lump-like grayish white coagulating necrotic area was formed; there was a definite border between this area and the normal tissue of liver, but the shape of the area was irregular; 24 hours later, the coagulating necrotic area becomes relatively regular and had a spheroid shape. The average diameter values of the coagulating necrotic areas found immediately after and 24 hours after injection of hydrochloric acid solutions of the following concentrations are shown in Table 1: 1.8 wt %, 3.6 wt %, 7.2 wt %, 10.8 wt %, 14.4 wt %, 18 wt %, and 25.2 wt %. According to Table 1, the diameter value of the coagulating necrotic area increases as the concentration of hydrochloric acid increases.

TABLE 1
The coagulating effects produced by 1 ml of hydrochloric acid of
different concentrations:

Diameter of coagulated area (cm)

| Concentrations of | immediately after | 24 hours after |
|---|---|---|
| Hydrochloric acid (%) | injection | injection |
| 1.8 | 0.7 | 0.75 |
| 3.6 | 1.0 | 1.4 |
| 7.2 | 1.3 | 1.9 |
| 10.8 | 1.5 | 2.2 |
| 14.4 | 1.8 | 3.1 |
| 18 | 2.2 | 4.8 |
| 25.2 | 2.4 | 5.2 |

Table 2 shows the average diameter values of coagulated ranges of liver tissues after the injection of pure hydrochloric acid in different doses to the liver tissues. According to table 2, as the dose of hydrochloric acid increased, the coagulated range and necrotic area of liver tissue were enlarged. Relatively good coagulating effects could be obtained by using small dose of pure hydrochloric acid. By comparing Table 1 with Table 2, the coagulated range resulting from the injection of 0.1 ml of analytic hydrochloric acid was 1.5 cm, which corresponded to the coagulated range resulting from the injection of 1.0 ml of hydrochloric acid with concentration of 3.6 wt %. (1.4 cm).

TABLE 2
The coagulating effects on liver tissues produced by analytic pure
hydrochloric acid (36% in concentration) in different doses.

Diameter of coagulated areas (cm)

| | immediately after | 24 hours after |
|---|---|---|
| Amount injected(ml) | injection | injection |
| 0.1 | 0.2 | 1.5 |
| 0.5 | 1.5 | 3.9 |
| 1.0 | 2.8 | 5.5 |

B. Pathologic Experiment Using Hydrochloric Acid to Coagulate Pig Liver in vivo

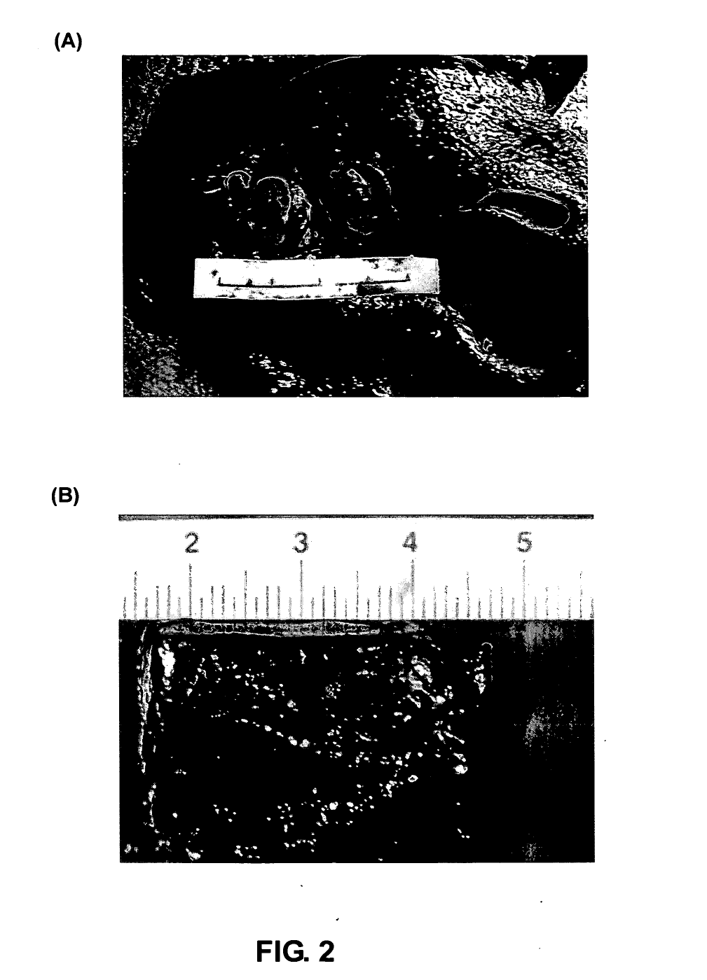

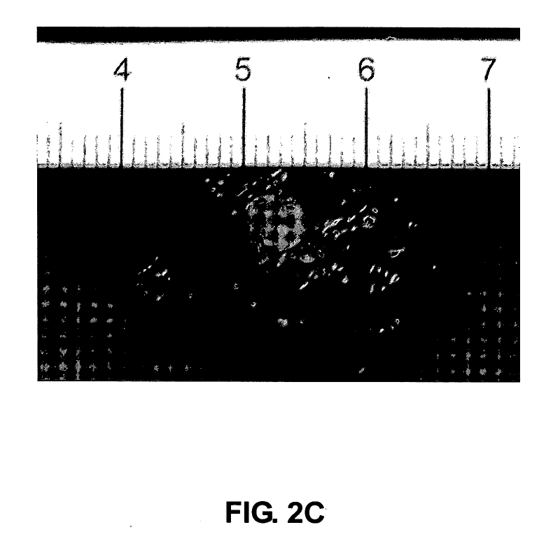

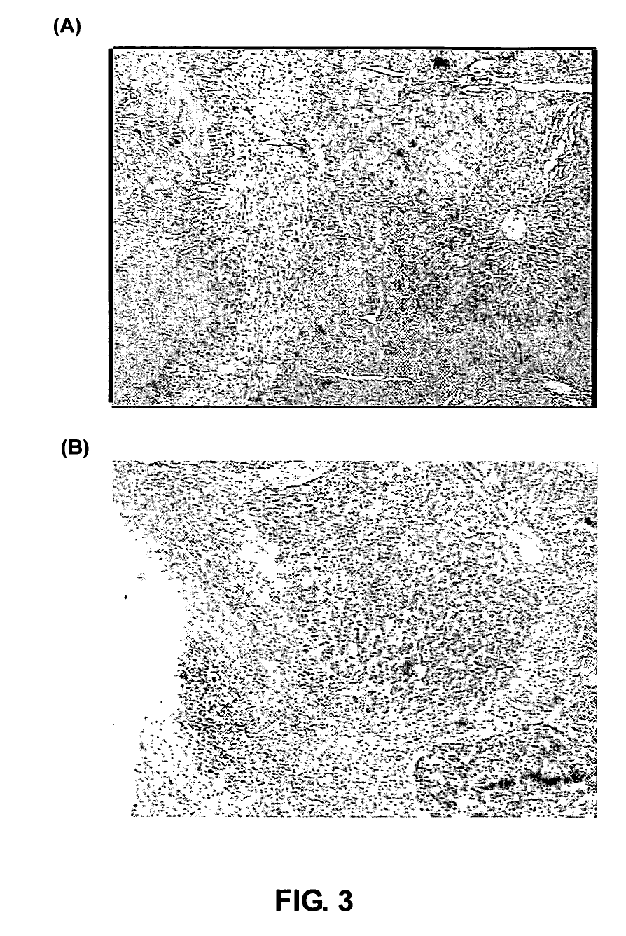

Ten hogs (females and males), with body weights of 70 kg on average, were fed in an enclosed area. The hair on the location of the liver of each hog was shaved and the skin sterilized guided by B-type ultrasonography. The puncture needle was punctured through the skin to the liver and then the needle was connected with an automatic micro-injecting pump. 1 ml of 3.6 wt % hydrochloric acid solution was injected slowly, at a speed of 0.3 ml/min, into the liver. Immediately after injection, it was found by B-type ultrasonography that the coagulated site had a round high level echo region, 2.0 cm in diameter. The animal was dissected at 1 week, 4 weeks, 12 weeks and 24 weeks after injection, and the shapes of the coagulating necrotic areas were observed and the diameters of the areas were measured. The results were showed by Table 3 and FIG. 2A-2C. Then the liver tissue including normal tissue and coagulating necrotic tissue was fixed with formalin. Paraffin sections were prepared and the histopathological changes were observed under a microscope. The results are shown by FIG. 3a-b. The results found at 1 week after injection were as follows: the coagulating necrotic range was 1.8 cm; the gross specimen showed that the border between coagulating necrotic liver tissue and normal liver tissue was clear, (see FIG. 3a). Under a microscope, the structure of coagulating necrotic liver tissue was divided into two layers: the necrotic area in the center, and the surrounding reacting area. The necrotic area occupied the most part of the tissue, where the structures of cells had completely disappeared and was replaced by a kind of evenly spreading, pale red necrotic tissue. Infiltration of a few lymphocytes and neutrophilic granulocytes could be found. The reacting area was a relatively thin reacting band between the necrotic and normal tissues. Proliferation of fibrous tissues was mainly found in this area (see FIG. 3A). The results found after 4 weeks after the injection were as follows: the coagulating necrotic range was 1.3 cm; the pathologic examination of the coagulating necrotic liver tissue showed that the necrotic area became smaller. Infiltration of large amount of lymphocytes and neutrophilic granulocytes occurred and proliferation of fibrous tissue was found, (see FIG. 3B).

The results found 12 weeks after injection were as follows: the coagulating necrotic range was 0.5 cm (see FIG. 3B). The results mentioned above are shown by Table 3. During the experiment, no animal died due to the treatment. Sixteen weeks after injection, the necrotic substances were absorbed. By dissection of the animal, it was difficult to find any sign of the treatment. The activities of all the animals were good.

TABLE 3
The coagulating effects produced by 3.6% of 1 ml hydrochloric
acid on pig liver in vivo

| | diameters of | |
|---|---|---|
| Time | coagulated | |
| (weeks) | areas(cm) | pathological changes |
| 1 | 1.8 | necrotic area occupies most part of the area, |
| | | the cell structure disappeared completely, the |
| | | reacting area was a reacting band between the |
| | | necrotic and the normal areas, proliferation of |
| | | fibrous tissue was mainly present in this area. |
| 4 | 1.3 | The necrotic area became smaller, infiltration |
| | | of large amount of lymphocytes and neutrophilic |
| | | granulocytes, and proliferation of fibrous tissue |
| | | could be found. |
| 12 | 0.5 | The necrotic area became further smaller, the |
| | | proliferation of fibrous tissue and formation of |
| | | scar occurred. |

C. The Comparison Among the Coagulating Effects on Pig Livers Produced by Hydrochloric Acid (HCl), Acetic Acid, and Absolute Alcohol.

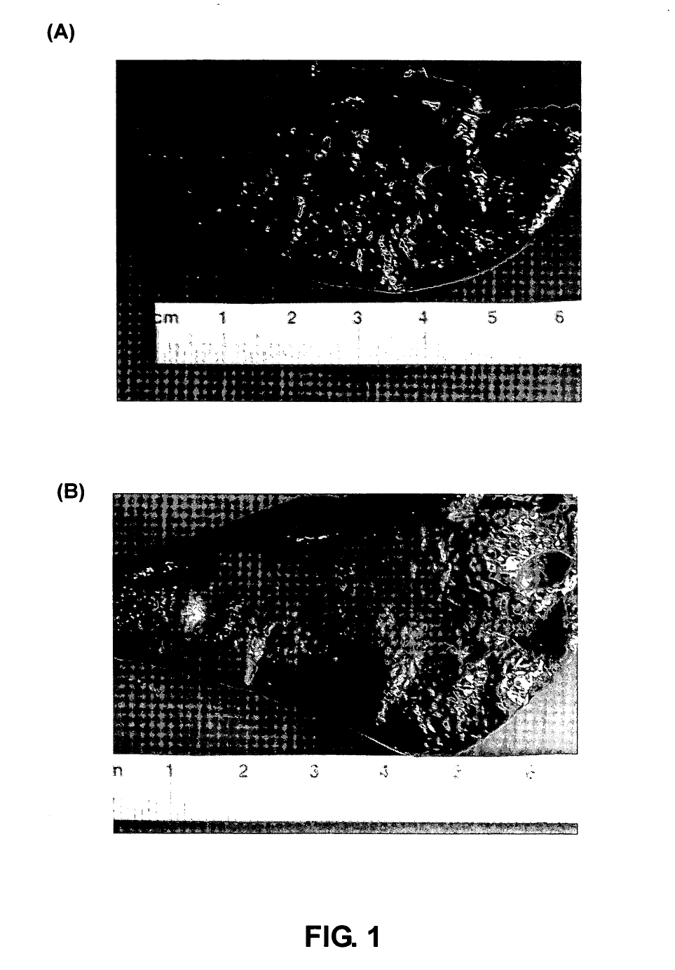

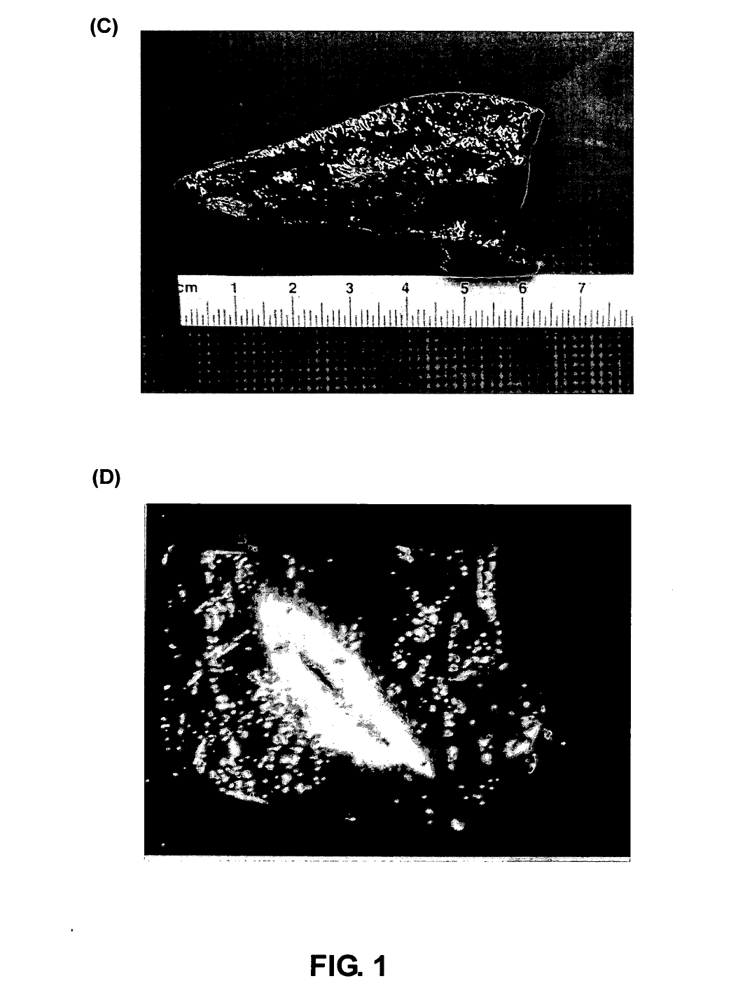

Twelve pig livers purchased from the market were divided into 4 groups, 3 in each group. A puncture needle was punctured to the center of the liver tissue and then connected with an automatic microinjecting pump. 1.8 wt % of HCl, 50% acetic, and 100% of absolute alcohol (0.5 ml each) were injected separately into the 3 groups of pig liver tissues slowly, at a speed of 0.3 ml/min. After 24 hours, the results of gross examinations showed that the coagulating necrotic area caused by the action of 18 wt % HCl was spheroid in shape. The average value of diameters was 2.2 cm, the necrotic area was grayish white, its color being even, the border between the necrotic area and the normal tissue was clear. The coagulating necrotic area caused by the action of 50% acetic was lump-like, 2.0 cm in diameter, its color being white but not even, the border between this area and the normal area was not clear. The coagulating necrotic area caused by the action of 100% absolute alcohol was spherical, white in color, 0.5 cm in diameter, the border between this area and the normal area was relatively clear. The results were showed in Table 4. The above results showed that the coagulating effects on pig liver in vitro produced by 18 wt % HCl was markedly better than those produced by the same dose of 50% acetic acid or 100% absolute alcohol. FIG. 1a-1C illustrated the coagulating effects on pig livers in vitro produced respectively by 0.5 ml of 18 wt % HCl, 0.5 ml of 50% acetic acid, and 0.5 ml of 100% absolute alcohol. Further, the coagulating effects of HCl on liver were also better than those produced by the existing technique—micro-wave coagulation. FIG. 1D shows the result of the experiment on the 4^{th} group using micro-wave 60W to act on pig livers for 60 s. The coagulating necrotic area produced in the location of action was olive-like, its border being clear. But the range of micro-wave coagulation was limited; the largest range being 2.5×1.5 cm, which means that the coagulation effect of HCl was better than that of micro-wave.

TABLE 4
The comparison between the coagulating effects on pig livers in
vitro produced by HCl, acetic acid, and absolute alcohol

| injected agent | diameter(cm) | gross shape |
|---|---|---|
| 18% HCl | 2.2 | spherical, grayish white in color, the |
| | | border between the necrotic and normal |
| | | areas was clear, |
| 50% acetic acid | 2.0 | lump-like, white, the border |
| | | between the necrotic and normal |
| | | areas was not clear |
| 100% absolute | 0.5 | spherical, white, the border |
| alcohol | | between the necrotic and normal areas |
| | | was clear |

Experiment in vivo: Nine hogs (females and males) with average body weight of 70 kg were fed in an enclosed area. The hogs were divided into 3 groups, 3 hogs in each group. The hair on the location of liver was shaved and the skin was sterilized. Guided by B-type ultrasonography, a puncture needle was punctured through the skin into the liver tissue and then connected with an automatic micro-injecting pump; 18 wt % of HCl, 50% acetic acid, and 100% absolute alcohol (1.0 ml each) were injected separately into the liver slowly, at a speed of 0.3 ml/min. The animals were dissected 1 week after the injection. The results of gross examinations were as follows: the coagulating necrotic area caused by the action of 18 wt % HCl was spheroid, the average diameter value being 2.1 cm, the necrotic area was grayish white, even in coloration, the border between this area and the normal area was clear. The coagulating necrotic area caused by the action of 50% acetic acid did not have a definite border, it was about 1.8 cm in diameter; the coagulating necrotic area caused by the action of 100% absolute alcohol was spherical, white in color, 0.5 cm in diameter. There was a relatively clear border between this area and the normal tissue.

The results described above showed that both in vitro and in vivo, the coagulating necrotic effects produced by HCl were better than that produced by acetic acid or absolute alcohol.

D. Pathologic Experiments Using HCl to Coagulate the Lungs of Hogs

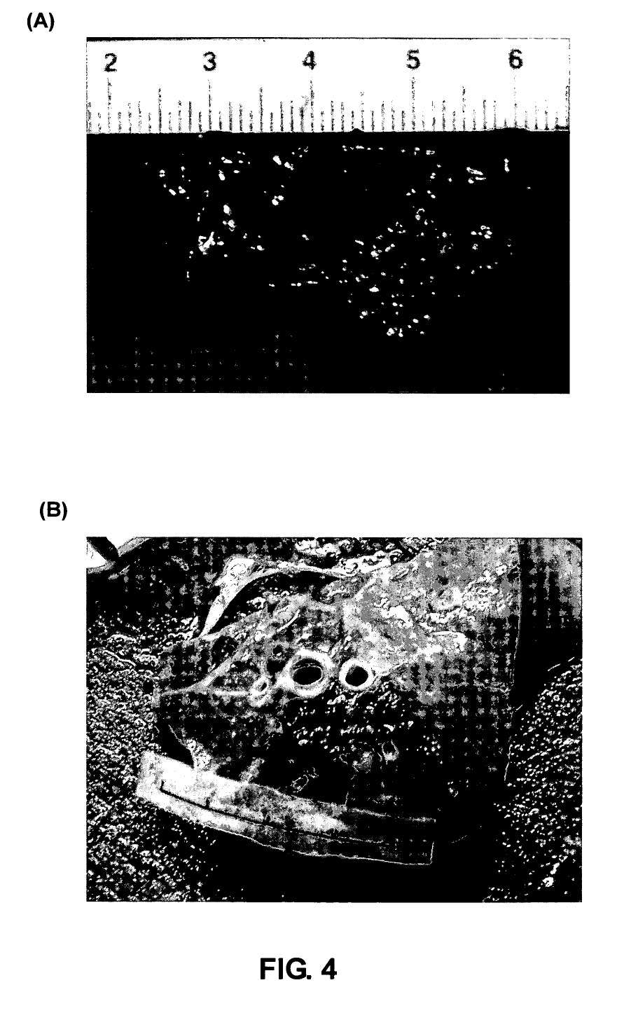

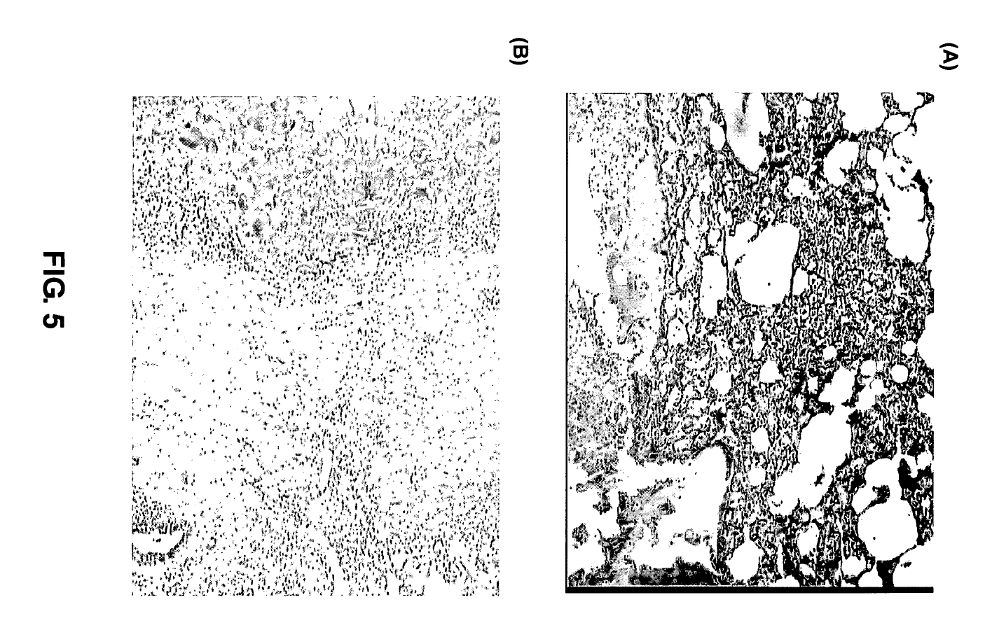

Four hogs (females and males) with average body weight of 70 kg were fed in an enclosed area. A puncture needle was punctured through the skin into the lung of the hog and then connected with an automatic micro-injecting pump; 1.0 ml of 3.6 wt % HCl was injected into the lung slowly, at a speed of 0.3 ml/min. At that time, the animal coughed slightly and the coughing stopped several minutes later. The lung of the animal was dissected 1 week after the injection. Gross examinations found that the color of the coagulated lung tissue was dark red when compared with the normal lung. The quality of the lung showed consolidation, being 1.8 cm in diameter, with a smooth capsule (see FIG. 4A). Four weeks after injection, dissection of the lung showed that the coagulating necrotic area became smaller (see FIG. 4B). A reacting band could be found between the necrotic and normal areas, the structure of pulmonary alveoli disappeared completely and was replaced by even, pale red necrotic tissue and large amount of red blood cells; infiltration of a small amount of lymphocytes, neutrophilic granulocytes could be found; the center of necrotic tissue was dry and old iron containing hemoglobin could be found (see FIGS. 5A, 5B). Three months after injection, the necrotic substances were absorbed, proliferation of fibrous tissue occurred. The coagulating tissue would be absorbed by the organism and some scar tissue was left. It was very difficult to find the coagulated focus by dissection.

E. Pathologic Experiment Using HCl to Coagulate Tumors of Rats in vivo.

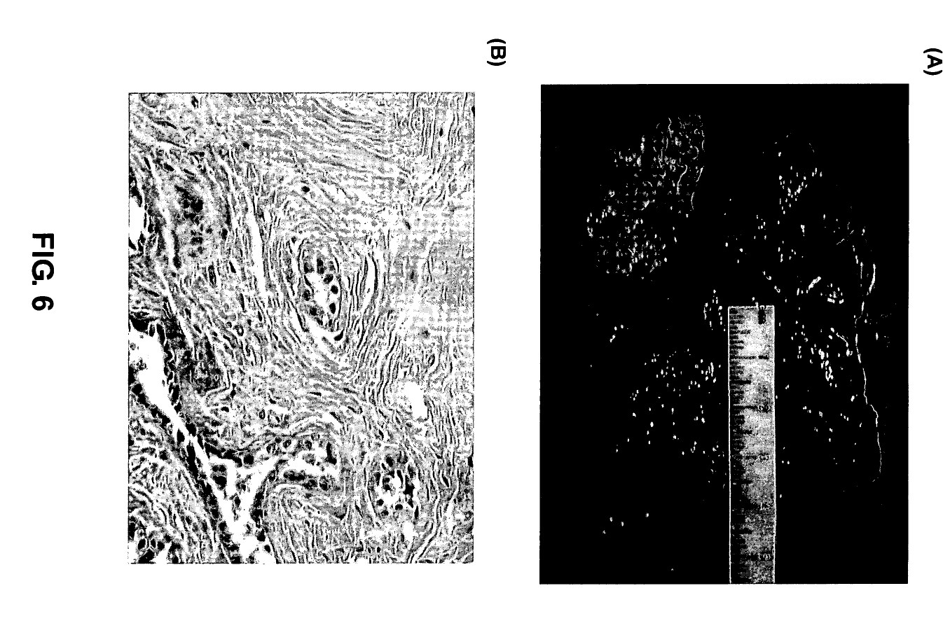

Rats, female and males, 500 g each in body weight, were fed routinely. Hypodermic implantation of W256 sarcoma was carried out. Sarcoma, 5 cm in diameter, was chosen. One (1.0) ml of 18 wt % HCl was slowly injected into the tumor. Twenty-four hours after injection, the rats were dissected, and the tumors were observed. Gross examination showed that the pale red tumor tissue had been turned into brown necrotic tissue after being coagulated by the medicament, its diameter being 3.5 cm; (see FIG. 6A). Through pathologic examination of the tumor, coagulating necrosis of tumor cells and infiltration of a small amount of inflammatory cell were found. During the treatment, no death or intoxication occurred among the rats. FIG. 6B illustrates the histopathologic changes found by microscopy, which shows that complete necrosis occurred in the necrotic area and that the border of the necrotic area was clear. The above findings show that HCl has coagulating effects on the tumors of rats.

F. The Comparison Among the Coagulating Effects Produced by HCl, Acetic Acid and Absolute Alcohol on Sarcomas of Rats.

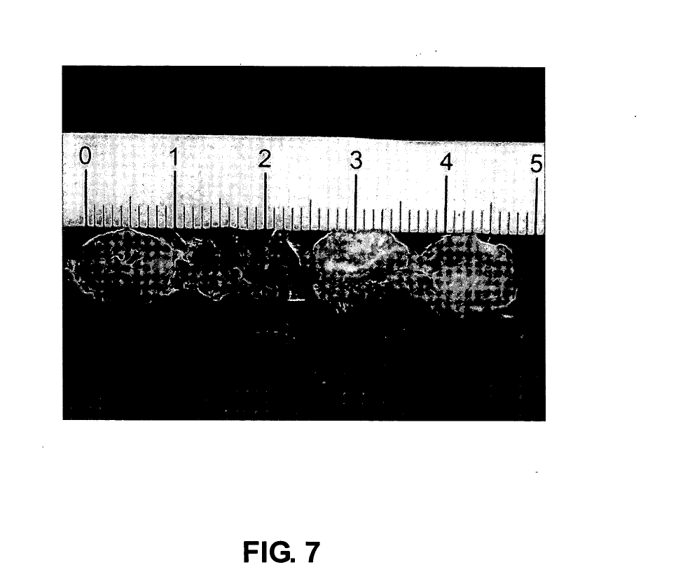

S-180 sarcomas were implanted into the hypodermal tissues of mice of Kun Ming species. When the tumor grew into 1 cm in size, a puncture needle was punctured into the center of the tumor and then connected with an automatic micro-injecting pump. 18 wt % HCl, 50% acetic acid, and 100% absolute alcohol (0.05 ml each) were injected separately and slowly, at a speed of 0.3 ml/min, into the tumor tissues. Forty-eight hours after injection, the results of gross examination (FIG. 7) were as follows: the coagulating necrotic area resulting from the action of HCl was spheroid in shape, 0.76 cm in diameter, the area showed even grayish white color and it was caseous, the border being clear (shown in FIG. 7). The coagulating necrotic area resulting from the action of 50% acetic acid was brown in color, 0.62 cm in diameter, its border being unclear (shown in FIG. 7). The coagulating necrotic area resulting from the action of 100% absolute alcohol was spherical, white in color, 0.41 cm in diameter, the border between the area and the normal tissue was relatively clear (shown in FIG. 7). The left 4 of FIG. 7 shows the results of the control group. The results of the actions of the three medicaments are showed by Table 5. The results show that HCl caused complete necrosis of the tumor, the border between the necrotic and normal tissue was clear; and the effects produced by HCl was better than those produced by acetic acid and absolute alcohol.

TABLE 5
The comparison between the coagulating effects produced by HCl,
acetic acid, and absolute alcohol (0.05 ml each) on sarcomas of rats

| | Diameters | |
|---|---|---|
| Agents injected | (cm) | Gross shapes |
| 3.6 wt % HCl | 0.76 | spherical, caseous, grayish white in color, |
| | | even, with clear border |
| 50% acetic acid | 0.62 | brown, the border was unclear, |
| 100% absolute | 0.41 | spherical, white in color, the border |
| alcohol | | between the necrotic and normal areas |
| | | was clear |

The results indicated that the range of tissue coagulated by HCl in vitro or in vivo was accurate. The coagulated tissue was spherical and that the coagulated range could be easily controlled by adjusting the concentration or dose of HCl.
G. Pathologic Experiment Using HCl to Coagulate the Skin of Guinea Pig

Six guinea pigs, female and males, 500 g each in body weight, were fed in the routine way. The guinea pig was anesthetized by ether. 0.2 ml of 3.6 wt % HCl was injected into the hypodermal tissue of the guinea pig. The results of injection were that the skin became rough and a little exudation occurred. One week after injection, surface of ulcer, 1.0 cm in diameter, was formed; iodine tincture was smeared on the surface. Three weeks later, the surface became dry and scar was formed, which indicated that HCl of low concentration produced relatively slight damaging effect without other adverse reaction.

H. Observation of the Antagonism of Sodium Bicarbonate Acting on the Coagulating Effect Produced by HCl

Thirty mice were used in the experiment. 0.05 ml of 3.8 wt % HCl was injected into the abdominal hypodermal tissue of each of them. At 0, 5, 10, and 20 minutes after the injection, 0.05 ml of 5% sodium bicarbonate solution was injected separately to 24 mice from the 30 mice. The injection of sodium bicarbonate was given to the site where HCl injection was given and the changes of the skin were observed. Injection of 0.05 ml of normal saline was given to each of the remaining 6 mice which served as the control group. Normal saline was injected into the corresponding site where HCl had been injected to the other experimental mice. The results were as follows: in the mice into which medicaments (HCl and sodium bicarbonate) were injected at the same time, no change was found in the skin; while in the mice of the control group, the experimental groups of 5 minutes, 10 minutes, 20 minutes, ulcer-like damages of the skin occurred and inflammatory exudates could be found. The diameters of damages being 1.2 cm, 0.8 cm, 0.9 cm, 0.8 cm. One week later, scar was formed in the necrotic area, the diameters of scar areas being 0.9 cm, 0.7 cm, 0.8 cm, 0.6 cm. By dissection, adhesion was found between the necrotic area and peritoneum. No damage was found in the abdominal organs, which indicated that the HCl used in this invention to prepare tumor-treating medicament could be detoxicated by an alkaline medicament.

I. The Toxic Effect on Pigs Produced by HCl

Twenty-seven hogs, raised in an enclosed area, were divided into 9 groups—3 hogs in each group. 18 wt % HCl was injected into the livers of the hogs at a rate of 0.1 ml/ min, 0.5 ml/min, 1 ml/min respectively, and the doses were 0.5 ml, 1 ml, 3 ml separately. One week after the injection, the animals were dissected. It was found that the coagulating necrotic ranges of the liver were 1.2-5.2 cm. No death or intoxication of hog occurred; which showed that when 18% HCl in the doses of 0.5 ml-3 ml was injected slowly into the liver of hog, no toxic effect was produced.

K. The Clinical Use of the Coagulation Produced by HCl on Liver Cancer

Four cases of primary liver cancer were chosen. The sizes of tumors were 2.4-3.0 cm. Before treatment the values of α-fetoglobulin were all higher than 400 μg/L (the normal value being <20 μg/L), the highest value was 1850 μg/L. After CT location, guided by CT, percutaneous puncture of the tumor was carried out with a 22G puncture needle, after the needle had hit the center of the tumor, it was connected with an automatic micro-injecting pump; 1.5 ml -2.0 ml of 18 wt % HCl was injected at a speed of 0.2 ml/min into each of the tumors; at the same time, 5 wt % sodium bicarbonate was given through intravenous infusion quickly to neutralize the blood. Immediately after the treatment, coagulated areas appeared, the average size of immediately coagulated ranges was 2.0×1.8 cm; 24 hours after injection, the coagulated range reached 3.2×3.0 cm. One week after injection, color ultrasonography showed that the tumor turned into high level echo without blood flow area. The liver cancer sensitive index—α-fetoglobulin value had been markedly lowered. None of the 4 patients had painful or toxic manifestation; the pH value of blood, the results of blood routine tests, results of liver and kidney function tests were all normal.

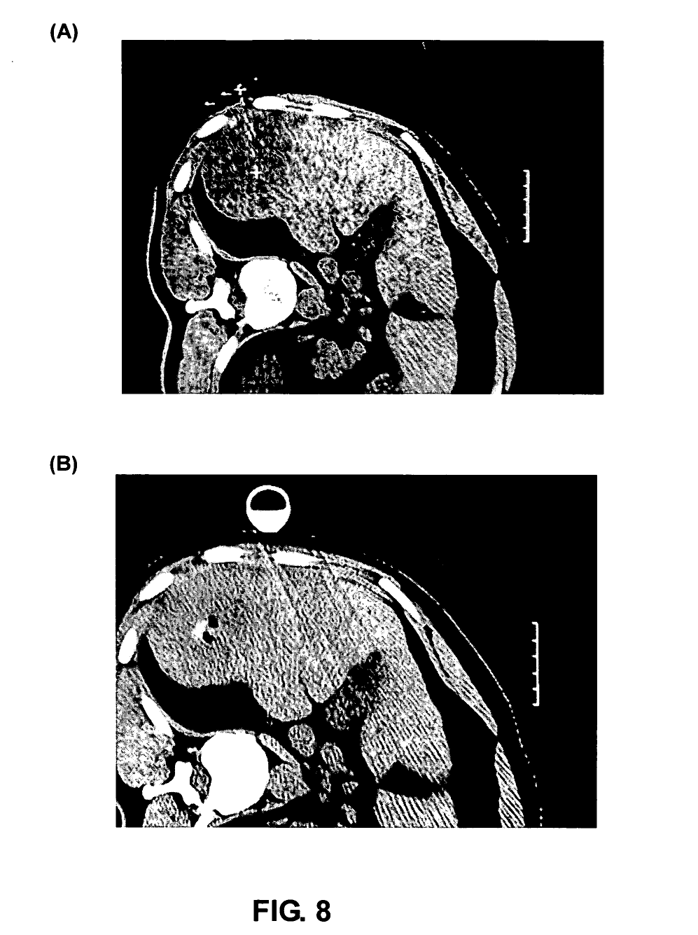

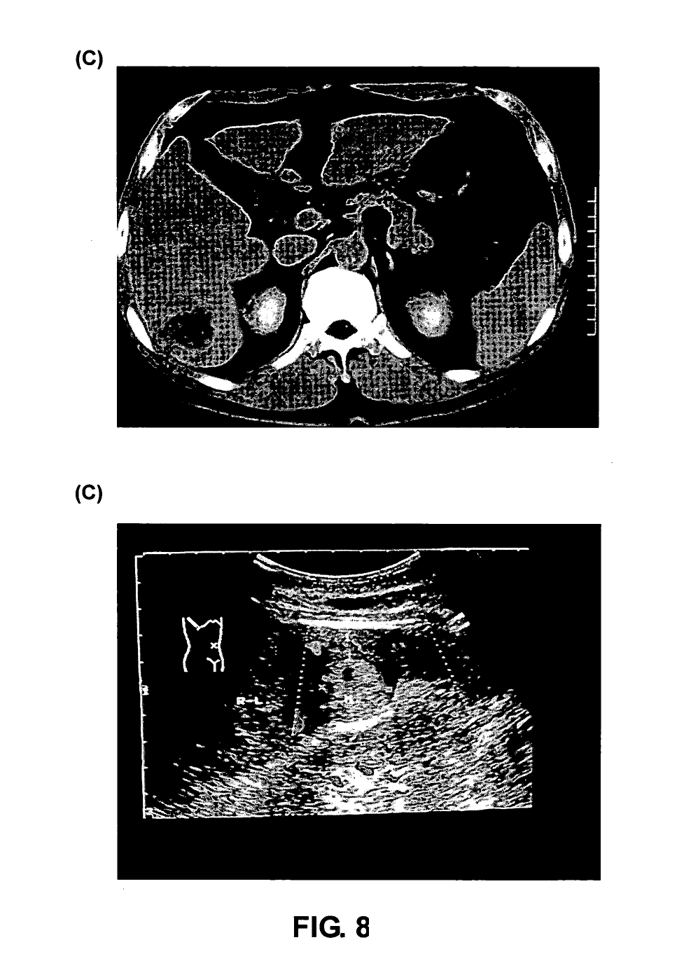

FIG. 8A shows a case of liver cancer, the diameter of the cancer was 3.0 cm, after CT location, the tumor was punctured, the needle hit the center of the tumor.

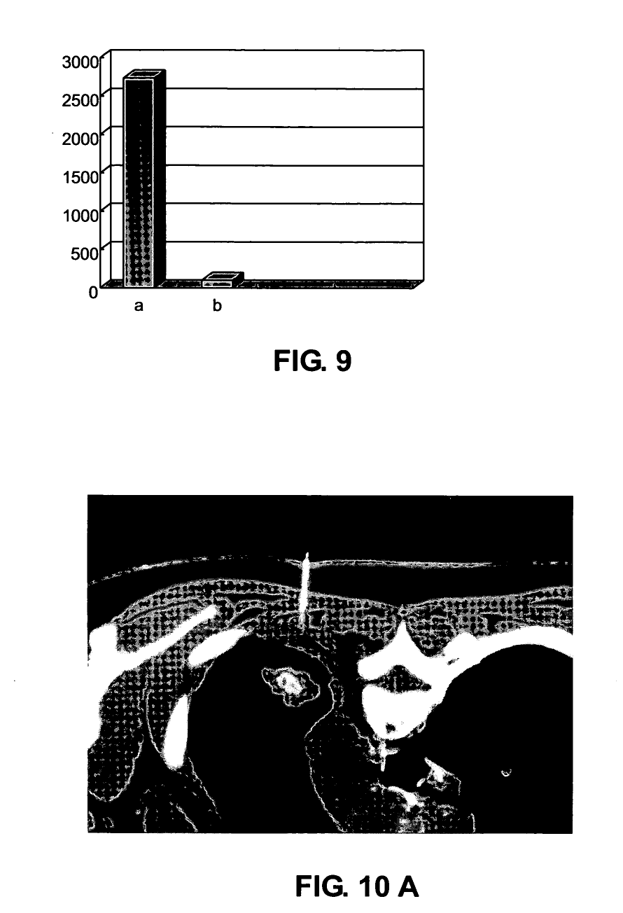

FIG. 8B illustrates that 2.0 ml of 18 wt % HCl was injected at a speed of 0.2 ml/min into the planned area; immediately after treatment, it could be seen that the medicament had been injected into the expected area and coagulated area appeared. FIG. 8C illustrates the intensified CT examination carried out on the said case of liver cancer at 24 hours after the injection of HCl, the necrotic range reached 3.5×3.0 cm. FIG. 8D showed the results of colored B-type ultrasonography carried out 1 week after injection; the tumor had turned into high level echo area without blood flow. One week after injection, liver cancer sensitive index—α-fetoglobulin values were markedly lowered; FIGS. 9A and 9B shows the difference between the α-fetoglobulin values before and after treatment.

L. The Clinical Use of the Coagulation Produced by HCl on Pulmonary Carcinoma

Two cases of primary bronchopulmonary carcinoma which were pathologically confirmed were chosen. One was a case of adenocarcinoma, the other was a case of adenous prickle cell carcinoma. The sizes of the two tumors were 2.0×2.0 cm and 2.5×2.0 cm respectively. After CT location, guided by CT, percutaneous puncture was carried out on each case; the needle reached the center of the tumor and connected with an automatic micro-injecting pump, 1.5, 2.0 ml of 18 wt % HCl were injected into the tumors at a speed of 0.2 ml/min. At the same time, 5% sodium bicarbonate injection solution was given through intravenous infusion quickly to neutralize the blood. Three days after treatment, the tumors of the two cases had basically disappeared. Cytological examination of needle puncture biopsy found necrotic tissue. The two patients coughed slightly during the treatment; no other painful or toxic manifestations occurred. pH value of blood, results of blood routine tests, results of liver and kidney function tests of the two patients were all normal.

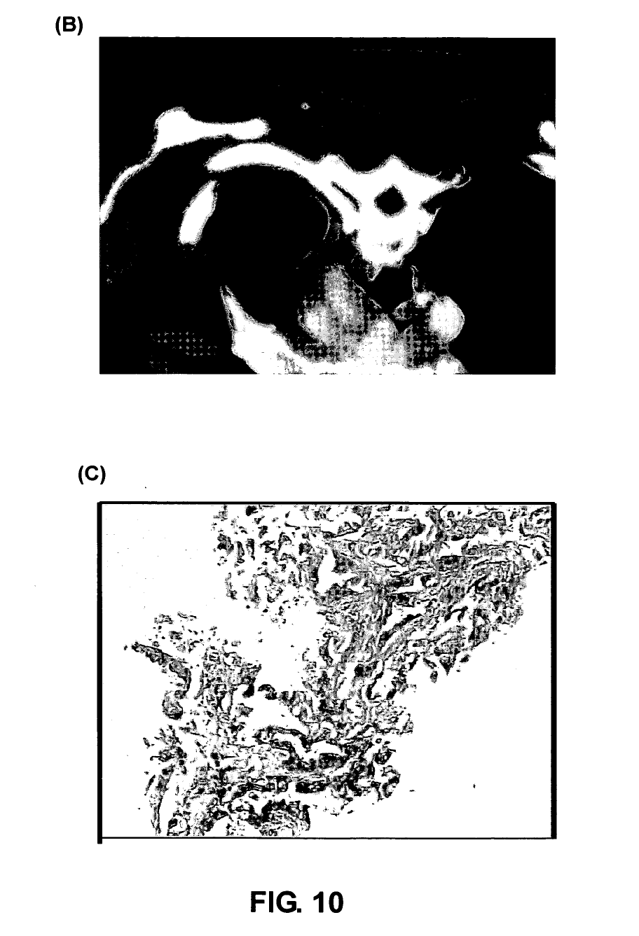

FIGS. 10A and 10B show respectively the results of CT examinations carried out immediately after and 3 days after 2.0 ml of 18% HCl was injected into the lung cancer whose diameter was 2.5×2.0 cm; it was found that immediately after injection, the medicament was injected to the planned area and coagulated area appeared; three days after injection, coagulating necrosis occurred in the tumor, the mass of tumor had basically disappeared. FIG. 10C shows the results of pathologic examination after needle-puncture biopsy, necrosis of tissue was found.

M. The Coagulating Effects Produced by HCl on Other Tumors

One case of recurrent carcinoma of breast, 2.0 cm×1.5 cm in diameter, was treated with 1.0 ml of 18 wt % HCl to coagulate the tumor; after being treated three times, the tumor was basically turned into necrotic tissue.

One case of metastatic tumor of the adrenal gland from pulmonary carcinoma was coagulated with 1.0 ml of 18 wt % HCl ; after being treated three times, the size of the tumor became 2.0 cm×2.0 cm in size, necrosis of the tumor occurred; the effects was relatively good.

One case of metastatic tumor of the left lobe of the brain from pulmonary adenocarcinoma, the size of the tumor being 1.9 cm×1.9 cm, was treated as follows: guided by CT, puncture was carried out with a 22G small needle through the skin and skull exactly into the center of the tumor, 1.0 ml of 18 wt % HCl was injected slowly to the tumor center to coagulate the tumor, the treatment was given two times; 3 months after treatment, re-examination done by intensified CT showed that the metastatic tumor had become small, being 0.8 cm in size.

Treatment of kidney cancer: guided by CT, the puncture needle hit the center of the tumor, 1.5 ml of 18 wt % HCl was injected into the tumor slowly. Before treatment, the size of the tumor was 3.0 cm×2.5 cm, 3 months after treatment, re-examination of the tumor showed that the tumor became a high level echo locus without blood flow, its diameter being 0.5 cm.

When the patients with cancerous pain due to advanced cancers were treated with 3.6 wt % HCl to block and damage the nerves, pain could be relieved.

The results disclosed herein show that the tissue-coagulating effects produced by HCl are definite and accurate. Under experimental conditions, HCl can bring about coagulation in tissues of the liver, lungs, and tumors. When HCl was used clinically to treat cancers of the liver, lungs, and metastatic carcinomas of the kidney, brain, adrenal gland, etc., good therapeutic effects were obtained. According to pathologic observation, the coagulating necrosis produced by HCl treatment was complete. The HCl effect is better than that produced by 50% acetic acid or absolute alcohol which is now used clinically. Because human gastric juice is made up of HCl, and no toxic reaction occurred during the experiments of this research work carried out on animals or in clinical practice, injection of HCl will not cause adverse effects on human bodies. Moreover, in normal tissues there are relatively many large blood vessels allowing the injected solution will be shunted. In the tumor tissues however, the blood vessels are mainly capillaries and when the medicament is slowly injected, the effect on tumor tissues should be better than that on the normal tissues.

Generally, the range of coagulation can be very easily controlled by adjusting the concentration or dose of HCl injected. For example, when 1 ml of 3.6 wt % to 18 wt % HCl or 0.1 ml-0.5 ml of analytic pure HCl is used to treat a tumor with a diameter less than 3 cm, the tumor will be completely coagulated and damaged. According to pathologic examinations, complete necrosis occurred in the coagulated tissue, and there was a clear border between the coagulated and normal tissues. Moreover, infiltration of lymphocytes and inflammatory cells could be found in the necrotic tissue, which was related to the increase of immune function and phagocytosis of necrotic tissue. Therefore, after coagulating necrosis of tumors, the necrotic substances were absorbed, proliferation of fibrous tissue occurred, the coagulated tissue would be absorbed by the organism and a small scar was left.

According to the above description, some of the advantages of this disclosure can be summarized as follows:

A new medical use of the already known chemical compound—HCl— has been developed.

Local injection of HCl disclosed herein does not produce adverse reaction or toxicity in human bodies. The pharmacological actions are strong, which indicates that the prospects of its being used widely are encouraging.

The source of material used in this invention is abundant, the cost of material is low, technique of preparation is simple and the material is used as a local injecting agent.

The medicament prepared with the material of this invention causes coagulating necrosis in tumor tissues, the therapeutic effects are definite, the coagulated range is accurate, spherical in shape, and is easily controlled. The medicament made in this invention can be used to treat any noumenal tumor, including primary malignant tumors, metastatic malignant tumors, and even some benign tumors; it can also be used to damage nerves to relieve the pain due to advanced cancers.

The effects produced by this invention are better than those produced by 50% acetic acid and 100% absolute alcohol which are used clinically now, and the effects can be detoxified by antagonists.

The method is further described with embodiment as follows.

### EXAMPLE 1

Analytic pure HCl bought from the market is diluted with sterile water for injection into 1.8% solution and put into 1 ml bottles. The bottles are then tightly sealed, sterilized; the solution is stored as a product.

### EXAMPLE 2

Analytic pure HCl bought from the market is diluted with sterile water for injection into 3.6% solution and put into 1 ml bottles. The bottles are then tightly sealed, sterilized; the solution is stored as a product.

### EXAMPLE 3

Analytic pure HCl bought from the market is diluted with sterile water for injection into 7.2% solution and put into 1 ml bottles, the bottles are then tightly sealed and sterilized and the solution is stored as a product.

### EXAMPLE 4

Analytic pure HCl bought from the market is diluted with sterile water for injection into 18% solution and put into 1 ml bottles. The bottles are then tightly sealed and sterilized and the solution is stored as a product.

1. The use of hydrochloric acid for the manufacture of a medicament for the curative treatment of tumors.

2. The use of hydrochloric acid according to claim 1, wherein the tumors are malignant tumors.

3. The use of hydrochloric acid according to claim 2, wherein the tumors are carcinomas of the liver, lungs, kidney, breast, or the metastatic carcinomas thereof, such as metastatic carcinomas of the brain, and adrenal gland.

4. The use of hydrochloric acid according to claim 1, wherein the tumors are benign tumors.

5. The use of hydrochloric acid for the manufacture of an analgesic for relieving cancerous pain.

6. The use of hydrochloric acid according to claim 1 wherein the concentrations of hydrochloric acid is about 1.8% to 36 wt %.

7. The use of hydrochloric acid according to claim 6 wherein the concentrations of hydrochloric acid is about 18 wt %.

8. The use of hydrochloric acid according to claim 7, wherein the dose of hydrochloric acid is about 0.05-5 ml.

9. The use of hydrochloric acid according to claim 2, wherein the concentrations of hydrochloric acid is about 1.8% to 36 wt %.

10. The use of hydrochloric acid according to claim 3, wherein the concentrations of hydrochloric acid is about 1.8% to 36 wt %.

11. The use of hydrochloric acid according to claim 4, wherein the concentrations of hydrochloric acid is about 1.8% to 36 wt %.

12. The use of hydrochloric acid according to claim 5, wherein the concentrations of hydrochloric acid is about 1.8% to 36 wt %.
